(19) United States
(12) Patent Application Publication
(10) Pub. No.: US 2025/0308642 A1
(43) Pub. Date: Oct. 2, 2025

(54) GENERATIVE SYSTEMS AND METHODS FOR PRODUCING CONFORMATIONAL ISOMERS USING HYBRID GENERATIVE ADVERSARIAL NETWORKS

(71) Applicant: BP Corporation North America Inc., Houston, TX (US)

(72) Inventors: Claudia Perry, London (GB); Clena Marie Abuan, Houston, TX (US); Corneliu Buda, Chicago, IL (US); Peter Lemke, Katy, TX (US); Omar Rodrigo Bacarreza Nogales, Cambridge (GB); William Riwal Clements, Cambridge (GB); Hugo Wallner, London (GB)

(73) Assignee: BP Corporation North America Inc., Houston, TX (US)

(21) Appl. No.: 19/092,220

(22) Filed: Mar. 27, 2025

Related U.S. Application Data
(60) Provisional application No. 63/570,403, filed on Mar. 27, 2024.

(51) Int. Cl. G16C 20/50; G06N 3/045; G16C 20/70

(52) U.S. Cl. CPC G16C 20/50; G06N 3/045; G16C 20/70

(57) ABSTRACT

A generative system for producing one or more conformers of a selected molecule includes a processor, and a memory coupled to the processor, wherein machine-readable instructions are stored in the memory, and wherein the machine-readable instructions, when executed on the processor, configure the processor to receive by a generative model both a training dataset and an input dataset, and generate by the generative model one or more synthetic conformers of the selected molecule using the input dataset associated with the selected molecule.

### CROSS-REFERENCE TO RELATED APPLICATIONS

The present application claims benefit of U.S. provisional patent application No. 63/570,403 filed Mar. 27, 2024, entitled “Generative Systems and Methods for Producing Conformational Isomers Using Hybrid Generative Adversarial Networks”, which is incorporated herein in its entirety for all purposes.

### STATEMENT REGARDING FEDERALLY SPONSORED RESEARCH OR DEVELOPMENT

Not applicable.

### BACKGROUND

Isomers are a fundamental concept in organic chemistry corresponding to molecules with identical molecular formulae (are of the same compound) but distinct structural arrangements of the atoms of the molecule in space. Several different types or classes of isomers exist including stereoisomers having the same connectivity of atoms for the given molecule and constitutional isomers that do not have the same connectivity of the atoms thereof. Stereoisomers include conformational isomers, often referred to as “molecular conformers” or simply “conformers,” which are a fundamental concept in organic chemistry that represent different arrangements of the same molecule resulting from the rotation of single bonds of the molecule.

### SUMMARY

An embodiment of a generative system for producing one or more conformers of a selected molecule comprises a processor, and a memory coupled to the processor, wherein machine-readable instructions are stored in the memory, and wherein the machine-readable instructions, when executed on the processor, configure the processor to receive by a generative model both a training dataset and an input dataset, and generate by the generative model one or more synthetic conformers of the selected molecule using the input dataset associated with the selected molecule. In some embodiments, the generative model comprises a generative-adversarial network (GAN). In some embodiments, the machine-readable instructions, when executed on the processor, further configure the processor to receive by a generator module of the GAN the training dataset, generate by the generator module the one or more synthetic conformers, receive by a discriminator module of the GAN the input dataset and the one or more synthetic conformers, and determine by the discriminator module an authenticity of the one or more synthetic conformers. In certain embodiments, the machine-readable instructions, when executed on the processor, further configure the processor to apply by the discriminator module a predefined target threshold or criteria to the one or more synthetic conformers received by the discriminator module from the generator module. In certain embodiments, the target threshold or criteria comprises a target energy level. In some embodiments, the target threshold or criteria comprises a target reactivity level. In some embodiments, the training dataset comprises a latent space from which information is provided to the generator module. In certain embodiments, the training dataset is at least partially obtained from or based on the input dataset. In certain embodiments, the training dataset is based on one or more authentic conformers of the selected molecule captured by the input dataset where one or more predefined parameters of the one or more authentic conformers have been masked from the generator module. In some embodiments, the one or more predefined parameters comprises one or more dihedral angles of the selected molecule. In some embodiments, the system comprises a quantum computing device that is configured to produce the latent space in the form of a quantum latent space. In certain embodiments, the quantum computing device comprises a simulated quantum processor. In certain embodiments, the quantum computing device comprises a photonic quantum computing device. In some embodiments, the processor and the memory comprise a classical computing device configured to implement the generator module and the discriminator module of the GAN whereby the GAN comprises a hybrid GAN.

An embodiment of a computer-implemented method for producing one or more conformers of a selected molecule comprises (a) applying a training dataset to a generative model to train the generative model to produce one more synthetic conformers of the selected molecule, and (b) producing by the generative model the one or more synthetic conformers of the selected molecule using an input dataset associated with the selected molecule. In some embodiments, the training dataset comprises a quantum latent space and the generative model comprises a hybrid GAN. In certain embodiments, the training dataset is based on one or more authentic conformers of the selected molecule captured by the input dataset where one or more predefined parameters of the one or more authentic conformers has been masked from a generator module of the hybrid GAN. In certain embodiments, (a) comprises applying a predefined target threshold or criteria to the one or more synthetic conformers received by a discriminator module of the GAN from the generator module.

An embodiment of a computer-implemented method for producing one or more conformers of a selected molecule comprises (a) receiving by a generative model an input dataset associated with the selected molecule, and (b) producing by the generative model one or more synthetic conformers of the selected molecule using the input dataset. In some embodiments, the generative model comprises a hybrid GAN.

Embodiments described herein comprise a combination of features and characteristics intended to address various shortcomings associated with certain prior devices, systems, and methods. The foregoing has outlined rather broadly the features and technical characteristics of the disclosed embodiments in order that the detailed description that follows may be better understood. The various characteristics and features described above, as well as others, will be readily apparent to those skilled in the art upon reading the following detailed description, and by referring to the accompanying drawings. It should be appreciated that the conception and the specific embodiments disclosed may be readily utilized as a basis for modifying or designing other structures for carrying out the same purposes as the disclosed embodiments. It should also be realized that such equivalent constructions do not depart from the spirit and scope of the principles disclosed herein.

### BRIEF DESCRIPTION OF THE DRAWINGS

For a detailed description of disclosed exemplary embodiments, reference will now be made to the accompanying drawings in which:

FIG. 1 is a block diagram of an embodiment of a generative system for producing conformers of one or more selected molecules in accordance with principles disclosed herein;

FIGS. 2 and 3 are block diagrams of another embodiment of a generative system for producing conformers of one or more selected molecules in accordance with principles disclosed herein;

FIG. 4 is a block diagram of another embodiment of a generative system for producing conformers of one or more selected molecules in accordance with principles disclosed herein;

FIGS. 5 and 6 are flowcharts of embodiments of methods for producing one or more conformers of a selected molecule in accordance with principles disclosed herein;

FIG. 7 is a block diagram of a computer system in accordance with principles disclosed herein.

### DETAILED DESCRIPTION

The following discussion is directed to various embodiments. However, one skilled in the art will understand that the examples disclosed herein have broad application, and that the discussion of any embodiment is meant only to be exemplary of that embodiment, and not intended to suggest that the scope of the disclosure, including the claims, is limited to that embodiment. The drawing figures are not necessarily to scale. Certain features and components herein may be shown exaggerated in scale or in somewhat schematic form and some details of conventional elements may not be shown in interest of clarity and conciseness.

In the following discussion and in the claims, the terms “including” and “comprising” are used in an open-ended fashion, and thus should be interpreted to mean “including, but not limited to . . .” Also, the term “couple” or “couples” is intended to mean either an indirect or direct connection. Thus, if a first device couples to a second device, that connection may be through a direct connection, or through an indirect connection as accomplished via other devices, components, and connections. In addition, as used herein, the terms “axial” and “axially” generally mean along or parallel to a central axis (e.g., central axis of a body or a port), while the terms “radial” and “radially” generally mean perpendicular to the central axis. For instance, an axial distance refers to a distance measured along or parallel to the central axis, and a radial distance means a distance measured perpendicular to the central axis. Additionally, the term “about” is intended to cover deviations of +/−5%.

As described above, conformers pertain to the different spatial arrangements or shapes that a given molecule can adapt. Conformers are a subset of stereoisomers, which are molecules with the same connectivity of atoms but differing spatial arrangements. Unlike configurational isomers (e.g., enantiomers and diastereomers), which require breaking and reforming chemical bonds to interconvert, conformers can interconvert rapidly by rotation around single bonds. Thus, different conformers of a single molecule may be produced or identified by rotating one or more single bonds of the molecule such as, for example, single carbon-carbon (C—C) bonds.

Generally, isomers are molecules with the same molecular formula but differing chemical structures or spatial arrangements. Conformers specifically arise due to rotation around single bonds, resulting in different three-dimensional arrangements of atoms. These different arrangements of the atoms of different conformers are energetically accessible and can influence the physical and chemical properties of the molecule. For example, organic molecules, particularly hydrocarbons, can exist in multiple conformations (three-dimensional shapes) due to the rotation of carbon-carbon single bonds. Different conformers may exhibit variations in dihedral angles, stability, reactivity, and other properties, which can significantly impact their utility in practical applications.

Dihedral angles refer to the angle between two planes in a given molecule, typically defined by four consecutive atoms. In the context of organic molecules like hydrocarbons, such as alkanes and cycloalkanes, rotation around C—C single bonds can lead to changes in dihedral angles between adjacently positioned carbon atoms. For example, in ethane, the eclipsed conformation has a dihedral angle of 0°, while the staggered conformation has a dihedral angle of 60°. These variations in dihedral angles can affect the molecule's stability and reactivity.

Particularly, the stability of different conformers is determined by their respective energy levels. In organic molecules, conformers with lower energy levels are typically more stable. For instance, in the case of ethane, the staggered conformation is relatively more stable than the eclipsed conformation due to the lower steric hindrance between hydrogen atoms of the staggered conformation. Understanding the stability of different conformers may be crucial in predicting their relative abundance and behavior under various conditions.

In addition, the reactivity of organic molecules can vary depending on their given conformation. Different conformers may exhibit distinct reactivity towards chemical reactions, such as substitution, addition, or elimination reactions. For example, in the case of cyclohexane, the chair conformation is more reactive than the boat conformation due to the differences in steric hindrance and bond angles. Assessing the reactivity of different conformers may play an important role in designing and optimizing chemical processes in hydrocarbon refining applications.

As an example, in hydrocarbon refining, the properties of organic molecules play a crucial role in determining the efficiency and effectiveness of refining processes. Conformers can significantly impact the behavior of hydrocarbon molecules during processes such as distillation, catalytic cracking, and hydroprocessing. Understanding the different conformers present in hydrocarbon feedstocks and products may therefore assist in optimizing refining processes to maximize product yields. For instance, in distillation processes, the separation of hydrocarbon mixtures is based on differences in boiling points and vapor pressures. Conformers with varying molecular weights and structural arrangements may exhibit different boiling points, leading to separation challenges. As another example, catalytic cracking is a process used in hydrocarbon refining to convert heavy hydrocarbons into lighter products such as gasoline and diesel. The presence of conformers in the feedstock can influence the selectivity and efficiency of cracking reactions. For instance, certain conformers may undergo preferential cracking pathways, leading to variations in product distributions and quality.

Different techniques have been conventionally utilized to produce and characterize conformers of molecules, such as organic molecules including hydrocarbons. For example, experimental techniques including nuclear magnetic resonance (NMR) spectroscopy, mass spectrometry, and X-ray crystallography have been employed for producing conformers of various molecules. In addition, computational techniques have been employed to produce and characterize conformers of different molecules. Computational methods, such as molecular mechanics and quantum mechanical calculations, have been used to predict the energetics and geometries of conformational isomers. Particularly, techniques that utilize energy minimization functions such as Density Function Theory (DFT) functions have also been employed to provide predictions of molecular structures, energies, and vibrational frequencies.

While computational methods like energy minimization-based methods may be used to study the stability and reactivity of organic molecules and their different conformers, such techniques are generally computationally intensive, limiting their applications to molecules having a limited number (e.g., twenty or fewer) of single bonds that may be rotated to produce different potential conformers of the molecule. However, for more complex molecules (e.g., molecules having twenty or more single bonds), the compute time for energy minimization-based methods is on the scale of days or weeks, too long for many commercial applications. Moreover, even when it is possible to produce possible conformations of a given molecule, it can be difficult to predict which of the identified conformations are the greatest importance or value.

Accordingly, embodiments of systems and methods for producing or identifying conformers of different molecules are disclosed herein which are substantially more computationally efficient than existing methods like energy minimization-based methods, allowing for the identification of different conformers of complex molecules having greater than 25 rotatable single bonds rotatable to produce different conformers. Particularly, instead of relying on energy minimization-based techniques, the generative system includes a generative model configured to produce, once trained on an existing conformer dataset, one or more conformations of one or more different molecules in a manner that is more computationally efficient than existing methods (e.g., requires fewer computational resources to produce the same conformers for the same molecule). As used herein, the term “generative model” refers to a computational model in which the generative model is trained by a training dataset to generate new or synthetic datasets using the training dataset.

In some embodiments, the generative model of the generative system includes a GAN. Generally, GANs include a generator module configured to generate “spurious” or synthetic data using random numbers drawn from a probability distribution referred to as the “latent space.” In addition, GANs include a discriminator module that receives both the synthetic data and “real” input data pertaining to a target object (e.g., a molecule of interest) and the synthetic data from the generator module. The discriminator module of the GAN is configured to “discriminate” or distinguish between the information contained in the authentic data and the information contained in the synthetic dataset. However, the generator module is configured to produce synthetic data that is indecipherable from the authentic data by the discriminator module. In other words, the generator module is trained to fool the discriminator module such that, at the conclusion of training, the discriminator module is unable to distinguish between the synthetic data and the real data. At this point, new, synthetic data may be created by the GAN via its trained generator module.

The configuration of the latent space used in conjunction with the generator module of the GAN may substantially impact the quality of the synthetic data produced by the GAN. For example, configuring the latent space to share features in common with the authentic data provided to the discriminator module may expedite training the of the GAN while allowing the GAN to produce higher quality synthetic data (e.g., more indistinguishable from the authentic data), including synthetic data in the form of one or more “synthetic” conformers (e.g., conformers created or generated by the generator module of the GAN) of a given molecule.

In some embodiments, the generative model of the generative system is a quantum-classical hybrid GAN (referred to herein as “hybrid GANs”) that leverages both quantum and classical computing. Quantum-classical hybrid GANs may provide modeling performance improvement with respective classical computing alone such as by, among other applications, generating plausible molecules. Such quantum-classical hybrid GANs may be simulate the operation of quantum hardware on classical computing devices or employ quantum hardware.

In certain embodiments, the generative system comprises a quantum computing device that produces or generates the latent space in the form of a quantum latent space used to provide random data to the generator module of the GAN. The advantages furnished by quantum computing relative to classical computing may be leveraged in this manner to generate conformers of substantially more complex molecules than would be practical for techniques implemented using only classical computing devices. Moreover, these advantages may be leveraged to generate conformers that are more accurate than those produced by generative systems implemented on classical computing devices.

As used herein, the term “quantum computing device” refers to a computing device configured to perform computing operations on information in the form of quantum bits or “qubits” and that leverage the unique properties of quantum mechanics including superposition and entanglement. Quantum computing devices are distinct from classical computing devices that process information in the form of “bits” and do not leverage the unique properties of quantum mechanics as with quantum computing devices. In certain embodiments, the quantum computing device comprises a photonic quantum computing device in which the qubits of the quantum computing device comprise photons. In certain embodiments, the quantum computing device (e.g., a photonic quantum computing device) is configured to implement the boson sampling model of quantum computation.

Thus, in at least some embodiments, a generative system comprises a generative model in the form of a hybrid GAN that utilizes a quantum latent space produced by a quantum computing device of the generative system. A generator module of the hybrid GAN may generate synthetic conformers of a selected molecule using information obtained from the latent space. In addition, a discriminator module of the hybrid GAN may determine the authenticity of the synthetic conformers produced by the generator module with respect to authentic information obtained from an input dataset associated with the selected molecule. In this manner, the generator and discriminator modules of the hybrid GAN may work in concert to train the generator module of the hybrid GAN to generate synthetic conformers that are indistinguishable by the discriminator module from the authentic information obtained from the input dataset. The trained generator module may now generate a plurality of synthetic conformers of the molecule with computing efficiency far in excess of conventional techniques. This may permit the generative system to rapidly generate conformers for complex molecules, including complex organic molecules such as hydrocarbons.

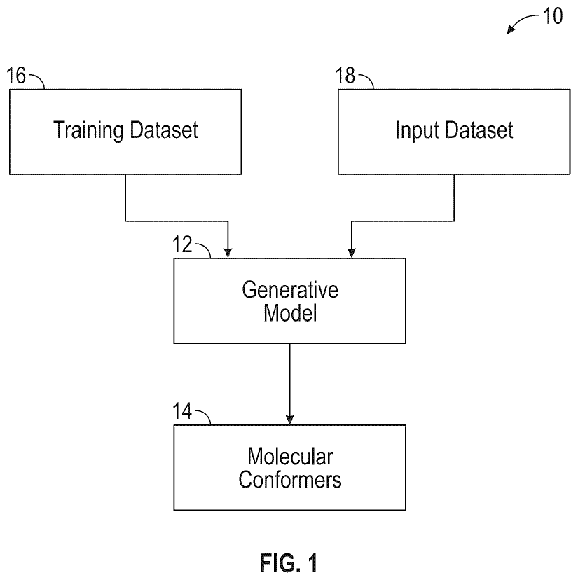

Referring now to FIG. 1, an embodiment of a generative system 10 for producing conformers for a selected molecule is shown. In this exemplary embodiment, generative system 10 includes a generative model 12 that produces or generates one or more conformers 14 of a selected molecule based on a training dataset 16 used to train the generative model 12 and an input dataset 18 provided to the generative model 12 once it has been trained using the training dataset 16.

Generally, the generative model 12 is trained by the training dataset 16 to generate molecular conformers 14 using the input dataset 18. In some embodiments, the molecular conformers 14 comprise synthetic molecular conformers initially created by the trained generative model 12 using the input dataset 18. In addition, the generative model 12 may utilize substantially fewer computing resources (e.g., computing processor power, computer memory) in order to generate the molecular conformers 14 for a given molecule relative to conventional techniques. Thus, the selected molecule associated with the molecular conformers 14 produced by the generative model 12 may be complex, having 25 or more single bonds rotatable to produce molecular conformers 14.

In some embodiments, the generative model 12 of generative system 10 comprises a GAN including a generator module and a discriminator module with training dataset 16 comprising a latent space from which information is provided to the generator module of the GAN. In other embodiments, generative model 12 may comprise models other than GANs. In certain embodiments, the generative model 12 comprises a hybrid GAN with training dataset 16 comprising a quantum latent space produced by a quantum computing device of the generative system 10. However, in other embodiments, generative model 12 can comprise a classical GAN that does not utilize a quantum computing device.

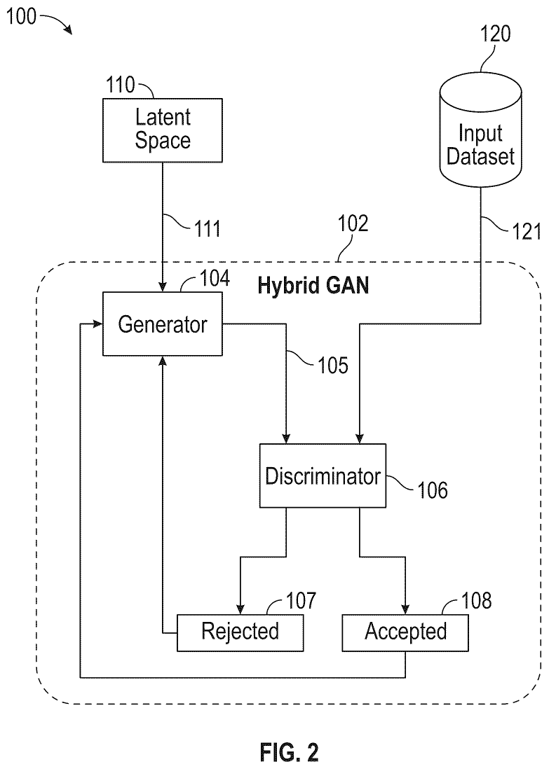

Referring to FIG. 2, an embodiment of a generative system 100 for producing conformers for a selected molecule is shown. In this exemplary embodiment, generative system 100 includes a GAN 102 shown in FIG. 2 in a training state. In some embodiments, the GAN 102 of generative system 100 includes a Wasserstein GAN such as a Wasserstein GAN with a gradient penalty (WGAN-GP); however, in other embodiments, the configuration or structure of GAN 102 may vary depending on the embodiment and requirements of the given application.

At least in the training state, GAN 102 includes both a generator module 104 and a discriminator module 106. Modules 104 and 106 comprise machine learning (ML) models such as neural networks (e.g., all-to-all connected graph neural networks where each node represents a different predefined parameter such as a dihedral angle of the conformer); however, the configuration of modules 104 and 106 may vary depending on the given embodiment and the requirements of the given application. Generator module 104 receives information from a probability distribution in the form of a latent space 110. Particularly, in this exemplary embodiment, generator module 104 receives random vectors 111 obtained or sampled from the latent space 110. Generator module 104 is configured to generate synthetic data 105 using the random vectors 111 sampled from the latent space 110. Particularly, the synthetic data 105 generated by generator module 104 is configured to mimic authentic data 121 obtained from an input dataset 120 that is associated with the selected molecule.

In some embodiments, the information contained in latent space 110 may share features or characteristics in common with input dataset 120 in order to improve the training speed and performance of the GAN 102. In some embodiments, information contained in latent space 110 and/or provided to generator module 104 may be based on information contained in input dataset 120. Particularly, in certain embodiments, information contained in the input dataset 120 (e.g., one or more conformers of the selected molecule) may be provided to the generator module 104 (e.g., through the latent space 110) where one or more predefined parameters of the provided information has been removed, masked, or hidden such that the information received by the generator module 104 is incomplete.

As an example, in certain embodiments, the authentic data 121 comprises one or more known, authentic conformers of the selected molecule and the information contained in the latent space 110 may be based on the known conformers contained in the authentic data 121. As an example, the generator module 104 of GAN 102 may receive as an input from latent space 110 a conformer of the selected molecule in which one or more predefined parameters of the conformer have been masked from the generator module 104 where the generator module 104 is configured synthetically to “fill in” this missing information. In some embodiments, the one or more predefined parameters comprise one or more dihedral angles of the conformer received by the generator module 104. In this manner, generator 104 may generate or return plausible, synthetic values for the masked dihedral angles (and/or other masked parameters in other embodiments) whereby synthetic conformers (including the synthetic dihedral angles) are generated by the generator module 104. In some embodiments, the modules 104 and 106 are invariant by permutation of the dihedral angles of the selected molecule. In certain embodiments, the architecture of GAN 102 is independent of the number of masked dihedral angles of the selected molecule so that training and inference may utilize different degrees of masking.

The discriminator module 106 of GAN 102 receives the synthetic data 105 from generator module 104 and compares it authentic data 121 obtained from input dataset 120 in an attempt to determine whether the synthetic data 105 is authentic or synthetic. For instance, discriminator module 106 may determine whether the synthetic data 105 corresponds to or matches the authentic data 121. In the training state of GAN 102, the discriminator module 106 may thus provide a first output in the form of rejected data 107 and a second output in the form of accepted data 108. Rejected data 107 corresponds to synthetic data 105 received by discriminator module 106 that the discriminator module 106 has determined to be synthetic or spurious and thus not matching or corresponding to the authentic data 121 received by discriminator module 106 from input dataset 120. Rejected data 107 and accepted data 108 are each returned to the generator module 104 as feedback data to assist the generator module 104 in more successfully fooling the discriminator module 106 whereby, over time, a greater proportion of the synthetic data 105 provided by generator module 104 to discriminator module 106 is (mistakenly) accepted by the discriminator module 106 as accepted data 108.

In certain embodiments, discriminator module 106 is configured to apply one or more predefined conditions or rules on the synthetic data 105 received by discriminator module 106 from generator module 104. At least some of these conditions may be configured to bias the discriminator module 106 towards only selecting as accepted data 108 synthetic data 105 that meets one or more predefined thresholds or criteria. These predefined thresholds may be associated with desirable properties of conformers of the selected molecule such that only conformers possessing these desirable properties are selected as accepted data 108 by the discriminator module 106. The predefined thresholds may be in the form of an inequality (e.g., the threshold only selects synthetic data 105 having a parameter that is equal to or greater than the predefined threshold, the threshold only selects synthetic data 105 having a parameter that is equal to or less than the predefined threshold) or in the form of a band (e.g., the threshold only selects synthetic data 105 having a parameter that falls within a predefined range or band of the threshold).

In this manner, discriminator module 106 may train the generator module 104 to generate, not only physically realistic conformers (e.g., ones indistinguishable from those contained in input dataset 120), but physically realistic conformers that possess one or more predefined, desired properties. As an example, in some embodiments, discriminator module 106 is configured to apply an energy threshold (e.g., a conformational energy threshold) whereby accepted data 108 comprises only conformers of the selected molecule meeting the energy threshold. Other salient parameters such as the reactivity of the conformer may also be applied in a similar manner to condition the resulting accepted data 108 produced by discriminator module 106. Utilizing thresholds such as the energy threshold discussed above permits the tuning of the conformers ultimately generated by the generative system 100. For instance, generative system 100 may be tuned to produce conformers having a low energy.

In some embodiments, modules 104 and/or 106 of GAN 102 are invariant by permutation of the masked parameter (e.g., the dihedral angles) given that the order in which the parameter is defined may not matter (e.g., it does not matter for dihedral angles). In addition, in certain embodiments, the architecture of GAN 102 is independent of the configuration or structure of the selected molecule (e.g., the number of dihedral angles of the molecule) so that training and inference can both use different types or levels of masking of the information provided to generator module 104.

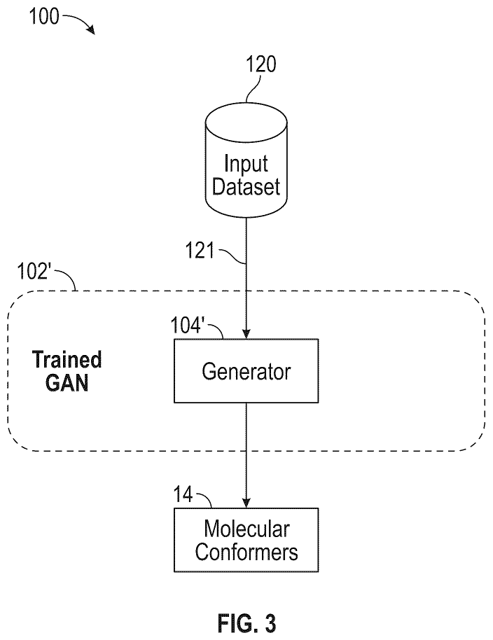

Referring to FIGS. 2 and 3, generative system 100 is shown in FIG. 3 with GAN 102′ in a trained or operational sate whereby the GAN 102. Particularly, over time, the discriminator module 106, in critiquing the work of the generator module 104, which improves or trains the generator module 104 to produce synthetic data 105 that is indistinguishable by the discriminator module 106 from the authentic data 121. Eventually, the generator module 104 produces data that is increasingly indistinguishable from authentic data 121, such that discriminator module 106 is no longer able to identify the synthetic data 107 received from generator module 104. At this point where generator module 104 has generally learned how to successfully fool the discriminator module 106, the GAN 102 transitions from the training state shown in FIG. 2 to the trained or operational sate shown in FIG. 3. In this configuration, the trained generator 104′ of trained GAN 102′ is configured to generate molecular conformers 14 of a selected molecule (e.g., an organic molecule such as a hydrocarbon) based on authentic data 121 provided by input dataset 120.

Moreover, by applying predefined target criteria or thresholds to the accepted data 108 during the training state of GAN 102, the molecular conformers 14 produced by the GAN 102′ may be tailored to possess one or more desirable properties such that only useful or otherwise targeted conformers of the selected molecule are created by generative system 100 rather than every plausible conformer of the selected molecule, thereby maximizing the efficiency of the generative system 200 (e.g., computing resources are not wasted on generating ultimately undesirable conformers).

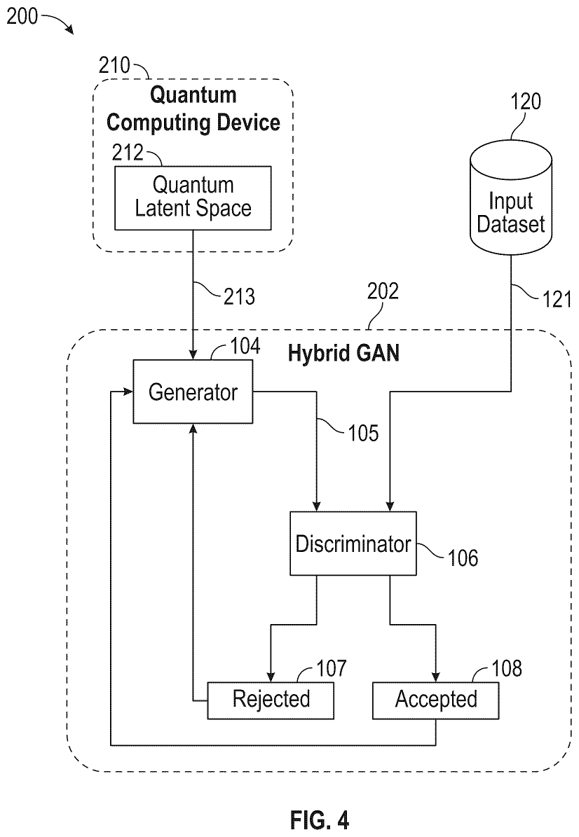

Referring to FIG. 4, an embodiment of a generative system 200 for producing conformers for a selected molecule is shown. In this exemplary embodiment, generative system 200 includes a hybrid GAN 202 including modules 104 and 106. In some embodiments, modules 104 and/or 106 of hybrid GAN 202 execute on a classical computing device or computer system.

In addition, generative system 200 includes a quantum computing device 210 that produces a quantum latent space 212 from which training data 213 (e.g., in the form of random vectors) is obtained or sampled and provided to the generator module 104. The quantum latent space 212 may be more complex in one or more respects than the classical latent space 110 shown in FIG. 2, which may facilitate superior performance of the generative system 200 (e.g., it may permit generator module 104 to produce broader classes of distributions) in some applications relative to the generative system 100 shown in FIGS. 2 and 3.

In some embodiments, the quantum computing device 210 comprises a simulated quantum processor such as a photonic computing device configured to implement the boson sampling method of quantum computation. For instance, quantum computing device 210 may comprise a linear optics quantum computation (LOQC) device, a photonic quantum computing (PQC) device), and the like. However, the structure, configuration, and/or operation of quantum computing device 210 may vary depending on the embodiment and the requirements of the given application. For example, in some embodiments, quantum computing device 210 may comprise quantum computing devices other than photonic quantum computing devices such as, for example, superconducting quantum computing devices, quantum dot computing devices, trapped ion computing devices, and the like.

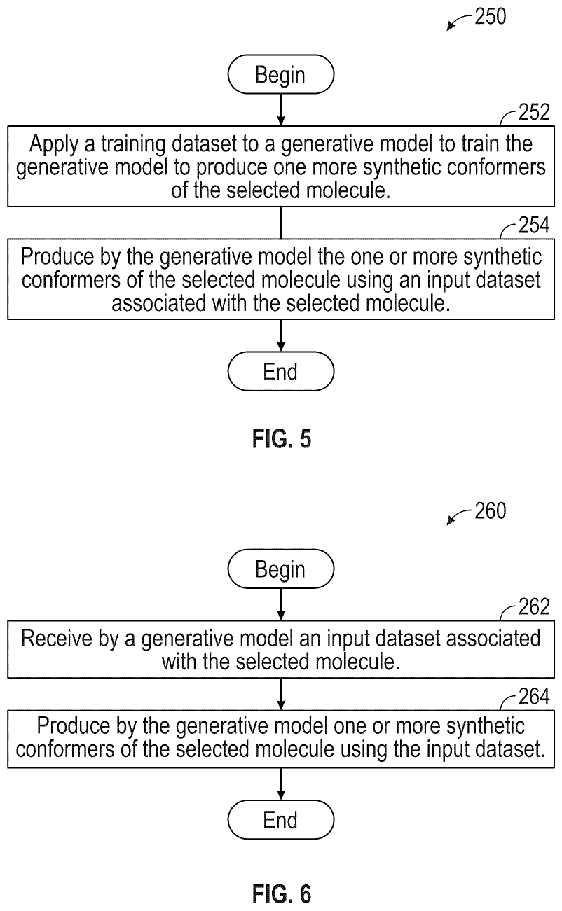

Referring now to FIG. 5, an embodiment of a method 250 for producing one or more conformers (e.g., molecular conformers 14 shown in FIGS. 1 and 3) of a selected molecule (e.g., an organic molecule such as a hydrocarbon) is shown. Initially, at block 252, method 250 comprises applying a training dataset (e.g., training dataset 16 shown in FIG. 1, random vectors 111 shown in FIG. 2, and training data 213 shown in FIG. 4) to a generative model (e.g., generative model 12 shown in FIG. 1, GAN 102 shown in FIG. 2, and hybrid GAN 202 shown in FIG. 4) to train the generative model to produce one more synthetic conformers (e.g., molecular conformers 14 shown in FIGS. 1 and 3) of the selected molecule. At block 254, method 250 comprises producing by the generative model the one or more synthetic conformers of the selected molecule using an input dataset (e.g., input dataset 18 shown in FIG. 1, input dataset 120 shown in FIG. 3) associated with the selected molecule.

Referring now to FIG. 6, an embodiment of a method 260 for producing one or more conformers (e.g., molecular conformers 14 shown in FIGS. 1 and 3) of a selected molecule (e.g., an organic molecule such as a hydrocarbon) is shown. Initially, at block 262, method 260 comprises receiving by a generative model (e.g., generative model 12 shown in FIG. 1, GAN 102′ shown in FIG. 3) an input dataset (e.g., input dataset 18 shown in FIG. 1, input dataset 120 shown in FIG. 3) associated with the selected molecule. At block 264, method 260 comprises producing by the generative model one or more synthetic conformers (e.g., molecular conformers 14 shown in FIGS. 1 and 3) of the selected molecule using the input dataset.

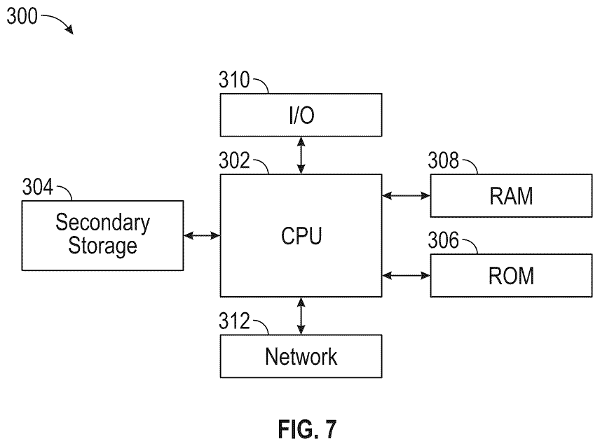

Referring now to FIG. 7, an embodiment of a computer system 300 is shown suitable for implementing one or more features disclosed herein. As an example, computer system 300 may be used to execute at least some of the features of generative systems 10, 100, and 200 shown in FIGS. 1-4, such as modules 104 and 106 of GAN 102 (shown in FIGS. 2 and 3) and hybrid GAN 202 (shown in FIG. 4). Particularly, in this exemplary embodiment, computer system 300 comprises a classical computer system that may execute the components of systems 10, 100, and 200 that are executed on classical computing devices.

The computer system 300 of FIG. 5 generally includes a processor 302 (which may be referred to as a central processor unit or CPU) that is in communication with memory devices including secondary storage 304, read only memory (ROM) 306, random access memory (RAM) 308, input/output (I/O) devices 310, and network connectivity devices 312. The processor 302 may be implemented as one or more CPU chips. It is understood that by programming and/or loading executable instructions onto the computer system 300, at least one of the CPU 302, the RAM 308, and the ROM 306 are changed, transforming the computer system 300 in part into a particular machine or apparatus having the novel functionality taught by the present disclosure.

Additionally, after the system 300 is turned on or booted, the CPU 302 may execute a computer program or application. For example, the CPU 302 may execute software or firmware stored in the ROM 306 or stored in the RAM 308. In some cases, on boot and/or when the application is initiated, the CPU 302 may copy the application or portions of the application from the secondary storage 304 to the RAM 308 or to memory space within the CPU 302 itself, and the CPU 302 may then execute instructions that the application is comprised of. In some cases, the CPU 302 may copy the application or portions of the application from memory accessed via the network connectivity devices 312 or via the I/O devices 310 to the RAM 308 or to memory space within the CPU 302, and the CPU 302 may then execute instructions that the application is comprised of. During execution, an application may load instructions into the CPU 302, for example load some of the instructions of the application into a cache of the CPU 302. In some contexts, an application that is executed may be said to configure the CPU 302 to do something, e.g., to configure the CPU 302 to perform the function or functions promoted by the subject application. When the CPU 302 is configured in this way by the application, the CPU 302 becomes a specific purpose computer or a specific purpose machine.

Secondary storage 304 may be used to store programs which are loaded into RAM 308 when such programs are selected for execution. The ROM 306 is used to store instructions and perhaps data which are read during program execution. ROM 306 is a non-volatile memory device which typically has a small memory capacity relative to the larger memory capacity of secondary storage 304. The secondary storage 304, the RAM 308, and/or the ROM 306 may be referred to in some contexts as computer readable storage media and/or non-transitory computer readable media. I/O devices 310 may include printers, video monitors, liquid crystal displays (LCDs), touch screen displays, keyboards, keypads, switches, dials, mice, track balls, voice recognizers, card readers, paper tape readers, or other well-known input devices.

The network connectivity devices 312 may take the form of modems, modem banks, Ethernet cards, universal serial bus (USB) interface cards, wireless local area network (WLAN) cards, radio transceiver cards, and/or other well-known network devices. The network connectivity devices 312 may provide wired communication links and/or wireless communication links. These network connectivity devices 312 may enable the processor 302 to communicate with the Internet or one or more intranets. With such a network connection, it is contemplated that the processor 302 might receive information from the network, or might output information to the network. Such information, which may include data or instructions to be executed using processor 302 for example, may be received from and outputted to the network, for example, in the form of a computer data baseband signal or signal embodied in a carrier wave.

The processor 302 executes instructions, codes, computer programs, scripts which it accesses from hard disk, floppy disk, optical disk, flash drive, ROM 306, RAM 308, or the network connectivity devices 312. While only one processor 302 is shown, multiple processors may be present. Thus, while instructions may be discussed as executed by a processor, the instructions may be executed simultaneously, serially, or otherwise executed by one or multiple processors. Instructions, codes, computer programs, scripts, and/or data that may be accessed from the secondary storage 304, for example, hard drives, floppy disks, optical disks, and/or other device, the ROM 306, and/or the RAM 308 may be referred to in some contexts as non-transitory instructions and/or non-transitory information.

In an embodiment, the computer system 300 may comprise two or more computers in communication with each other that collaborate to perform a task. For example, but not by way of limitation, an application may be partitioned in such a way as to permit concurrent and/or parallel processing of the instructions of the application. Alternatively, the data processed by the application may be partitioned in such a way as to permit concurrent and/or parallel processing of different portions of a data set by the two or more computers. In an embodiment, the functionality disclosed above may be provided by executing the application and/or applications in a cloud computing environment. Cloud computing may comprise providing computing services via a network connection using dynamically scalable computing resources.

While disclosed embodiments have been shown and described, modifications thereof can be made by one skilled in the art without departing from the scope or teachings herein. The embodiments described herein are exemplary only and are not limiting. Many variations and modifications of the systems, apparatus, and processes described herein are possible and are within the scope of the disclosure. Accordingly, the scope of protection is not limited to the embodiments described herein, but is only limited by the claims that follow, the scope of which shall include all equivalents of the subject matter of the claims. Unless expressly stated otherwise, the steps in a method claim may be performed in any order. The recitation of identifiers such as (a), (b), (c) or (1), (2), (3) before steps in a method claim are not intended to and do not specify a particular order to the steps, but rather are used to simplify subsequent reference to such steps.

Among other embodiments, hybrid quantum/classical generative modelling algorithm for generating low-energy conformations of small and medium size hydrocarbon molecules are described herein. The possible conformations of each molecule corresponds to its 3-dimensional shape, which determines several of its physical and chemical characteristics. Due to the large search space, traditional physical solvers struggle to find low-energy conformers for some of the tested molecules. To address this issue, we investigate the potential of using a hybrid GAN algorithm. We train a GAN that trains a hybrid quantum/classical generator using a photonic quantum processor and a GPU on a dataset of alkane molecules to generate conformers with a specified energy. This approach successfully generates molecules with the specified energy. Moreover, the hybrid generator significantly outperforms an equivalent classical generator at this task. This work shows that near-term quantum processors hold potential for improving the performance of machine learning algorithms in chemistry.”

What is claimed is:

1. A generative system for producing one or more conformers of a selected molecule, the system comprising:
a processor; and
a memory coupled to the processor, wherein machine-readable instructions are stored in the memory, and wherein the machine-readable instructions, when executed on the processor, configure the processor to:
receive by a generative model both a training dataset and an input dataset; and
generate by the generative model one or more synthetic conformers of the selected molecule using the input dataset associated with the selected molecule.

2. The system of claim 1, wherein the generative model comprises a generative-adversarial network (GAN).

3. The system of claim 2, wherein the machine-readable instructions, when executed on the processor, further configure the processor to:
receive by a generator module of the GAN the training dataset;
generate by the generator module the one or more synthetic conformers;
receive by a discriminator module of the GAN the input dataset and the one or more synthetic conformers; and
determine by the discriminator module an authenticity of the one or more synthetic conformers.

4. The system of claim 3, wherein the machine-readable instructions, when executed on the processor, further configure the processor to:
apply by the discriminator module a predefined target threshold or criteria to the one or more synthetic conformers received by the discriminator module from the generator module.

5. The system of claim 4, wherein the target threshold or criteria comprises a target energy level.

6. The system of claim 4, wherein the target threshold or criteria comprises a target reactivity level.

7. The system of claim 3, wherein the training dataset comprises a latent space from which information is provided to the generator module.

8. The system of claim 3, wherein the training dataset is at least partially obtained from or based on the input dataset.

9. The system of claim 7, wherein the training dataset is based on one or more authentic conformers of the selected molecule captured by the input dataset where one or more predefined parameters of the one or more authentic conformers have been masked from the generator module.

10. The system of claim 9, wherein the one or more predefined parameters comprises one or more dihedral angles of the selected molecule.

11. The system of claim 7, further comprising a quantum computing device that is configured to produce the latent space in the form of a quantum latent space.

12. The system of claim 11, wherein the quantum computing device comprises a simulated quantum processor.

13. The system of claim 11, wherein the quantum computing device comprises a photonic quantum computing device.

14. The system of claim 11, wherein the processor and the memory comprise a classical computing device configured to implement the generator module and the discriminator module of the GAN whereby the GAN comprises a hybrid GAN.

15. A computer-implemented method for producing one or more conformers of a selected molecule, the method comprising:
(a) applying a training dataset to a generative model to train the generative model to produce one more synthetic conformers of the selected molecule; and
(b) producing by the generative model the one or more synthetic conformers of the selected molecule using an input dataset associated with the selected molecule.

16. The method of claim 15, wherein the training dataset comprises a quantum latent space and the generative model comprises a hybrid generative-adversarial network (GAN).

17. The method of claim 16, wherein the training dataset is based on one or more authentic conformers of the selected molecule captured by the input dataset where one or more predefined parameters of the one or more authentic conformers has been masked from a generator module of the hybrid GAN.

18. The method of claim 17, wherein (a) comprises applying a predefined target threshold or criteria to the one or more synthetic conformers received by a discriminator module of the GAN from the generator module.

19. A computer-implemented method for producing one or more conformers of a selected molecule, the method comprising:
(a) receiving by a generative model an input dataset associated with the selected molecule; and
(b) producing by the generative model one or more synthetic conformers of the selected molecule using the input dataset.

20. The method of claim 19, wherein the generative model comprises a hybrid generative-adversarial network (GAN).
